(19) United States
(12) Patent Application Publication
(10) Pub. No.: US 2011/0257662 A1
(43) Pub. Date: Oct. 20, 2011

(54) System, Method and Apparatus for Hair Removal

(75) Inventors: Charlie Pfau, Logan, UT (US); Michelle Hancey, Murray, UT (US)

(73) Assignee: BRILLIANCE MARKETING INTERNATIONAL, LLC, Providence, UT (US)

(21) Appl. No.: 13/088,987

(22) Filed: Apr. 18, 2011

Related U.S. Application Data
(60) Provisional application No. 61/325,730, filed on Apr. 19, 2010.

(51) Int. Cl. A61B 17/50

(52) U.S. Cl. 606/133; 132/200

(57) ABSTRACT

Implementations of the present invention include systems, method and apparatus for hair removal. The hair removal system utilizing a reusable non-strip based member for applying, spreading and/or removing a hair removal compound. According to one aspect, a practitioner applies a sugar based natural wax to a target hair removal area, such that the sugar based natural wax can be warmed on contact with the users skin to bond with the hair to be removed and then quickly removed from the skin to remove not only the hair removal compound, but also hair which has bonded thereto. Implementations of the invention also include a reusable flexible member adapted to scoop, apply and/or remove the volume of hair removal compound with respect to a target hair removal area.

### CROSS-REFERENCE TO RELATED APPLICATIONS

The present invention claims the benefit of priority to U.S. Provisional Patent Application No. 61/325,730, filed Apr. 19, 2010, entitled “System, Method and Apparatus for Hair Removal,” the entire content which is incorporated by reference herein.

### BACKGROUND OF THE INVENTION

1. Field of the Invention

The present invention is directed to systems and methods for and apparatus for hair removal. In more particular the present invention is directed to a system utilizing a sugar based or other natural wax and utilizing a reusable removal apparatus to facilitate removal of hair in a target location.

2. Background and Relevant Art

FIG. 1 is a perspective view of a prior art wax system using disposable strips for removing hair. Hair removal wax systems are well known in the art and have been utilized for many years. Such systems suffer from a number of deficiencies. In the illustrated embodiment, a user 10 is shown utilizing the hair removal system to remove unwanted hair. In the illustrated embodiment, the user's leg 12 is the target location 14. An amount of hair 16 is depicted which the user desires to remove. An amount of wax 18 has been applied to the target location 14. Application of the wax can be applied utilizing various technologies and compositions, but traditionally involves warming the wax until it is somewhat liquid. The wax is then applied to the target location 14. Application of wax 18 to target location 14 and the resultant contact with the user's skin will typically result in cooling of wax 18 so as to somewhat solidify wax 18. In the process of cooling, wax 18 binds with exposed hair in target location 14.

A disposable strip 20 is applied to wax 18 as it cools, resulting in binding between wax 18 and disposable strip 20. As disposable strip 20 is lifted, often in a quick motion, disposable strip, wax 18 and hair 16 are removed from target location 14 leaving hairless and smooth skin. Because the root of the hair is typically removed from the follicle, growth of hair above the skin surface will typically take several days to several weeks. This prolonged period of lack of exposed hair is desirable both for the non-prickly feeling, but also for the increased duration of hairless cycles?

Drawbacks of such systems are fairly well known. First, removal strips must be purchased in quantity to allow for reapplications. As a result, unwanted cost and storage are needed. Additionally, to the extent that an amount of wax is left without remaining disposable strips, wax will be wasted. Alternatively, unused strips remain without remaining wax resulting in waste of the strips. Additionally, storage of the strips can be messy, unsightly and inconvenient. To the extent that storage with the wax is not desired, it is easy to lose one or both of the wax and strips leading to further displeasure with the system.

### BRIEF SUMMARY OF THE INVENTION

Implementations of the present invention provide systems, method and apparatus for hair removal. The hair removal system utilizing a reusable non-strip based member for applying, spreading and/or removing a hair removal compound.

According to one aspect, the hair removal system for use in removing unwanted hair by mechanical removal of the hair from the hair follicle, utilizing a sugar based natural wax which can be applied to a target hair removal area such that the sugar based natural wax can be warmed on contact with the users skin to bond with the hair to be removed and then quickly removed from the skin to remove not only the hair removal compound, but also hair which has bonded thereto, the hair removal system including a volume of hair removal compound configured to bond with hair upon application to the user's skin. A reusable flexible member adapted to scoop, apply and/or remove the volume of hair removal compound with respect to a target hair removal area.

A method for utilizing a hair removal system for use in removing unwanted hair by mechanical removal of the hair from the hair follicle, utilizing a sugar based natural wax which can be applied to a target hair removal area such that the sugar based natural wax can be warmed on contact with the users skin to bond with the hair to be removed and then quickly removed from the skin to remove not only the hair removal compound, but also hair which has bonded thereto, the hair removal system comprising. Obtaining a volume of hair removal compound configured to bond with hair upon application to the user's skin. Obtaining a reusable flexible member adapted to scoop, apply and/or remove the volume of hair removal compound with respect to a target hair removal area. Scooping a bolus of hair removal compound from the volume of hair removal compound. Applying the bolus of hair removal compound to the target hair removal area utilizing the reusable flexible member. Spreading the bolus of hair removal compound to form a layer substantially covering the target hair removal area. Removing the layer of hair removal compound utilizing the reusable flexible member. As necessary reapplying the same bolus of hair removal compound to a subsequent target hair removal area.

Additional features and advantages of exemplary implementations of the present invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims, or may be learned by the practice of such exemplary implementations as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 is a perspective view of a prior art wax system using disposable strips for removing hair;

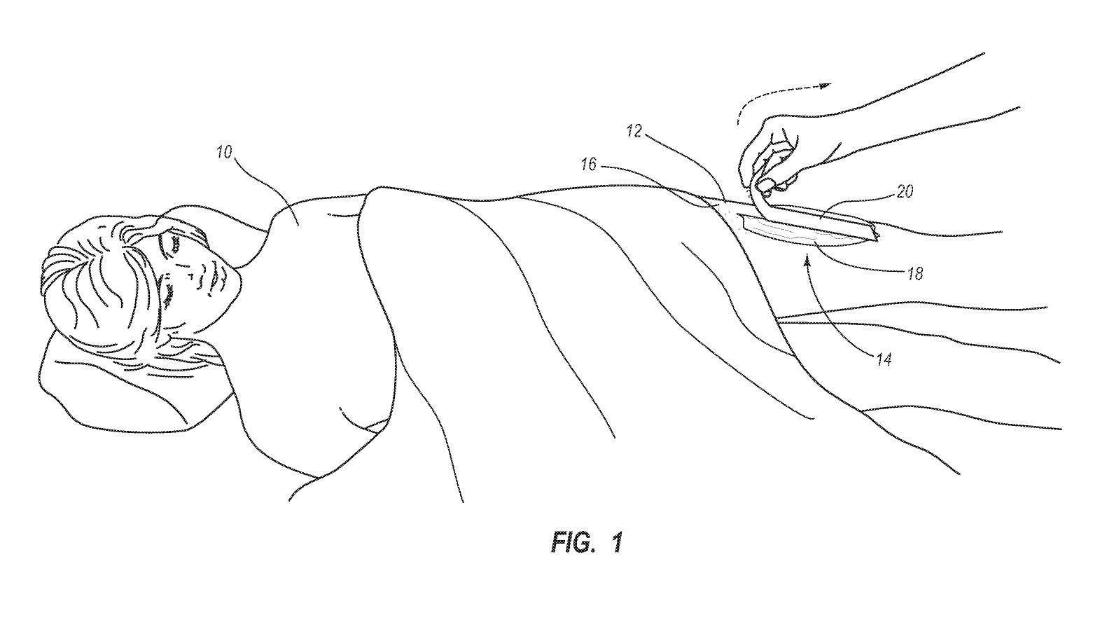

FIG. 2 illustrates components of the hair removal system of the present invention including reusable removal apparatus;

FIG. 3 illustrates a user obtaining a bolus of sugar based hair removal compound in preparation for employing hair removal methodology;

FIG. 4 is a perspective view illustrating a user applying hair removal compound to a target location for hair removal;

FIG. 5 illustrates a layer of removal compound that has been spread across the target location;

FIG. 6 depicts positioning of the reusable removal apparatus in preparation for removal of hair from the target area;

FIG. 7 illustrates removal of all or a portion of hair removal compound utilizing the reusable removal compound;

FIG. 8 illustrates a section of the user's body where hair has been removed utilizing the hair removal compound and the reusable removal apparatus;

FIG. 9 depicts a user's leg that has been treated resulting in complete hair removal;

FIG. 10 depicts a targeted reusable hair removal apparatus being utilized on a user's face; and

FIG. 11 illustrates a method for utilizing a hair removal compound with a reusable removal apparatus.

### DETAILED DESCRIPTION OF THE PREFERRED EMBODIMENTS

Implementations of the present invention provide systems, method and apparatus for hair removal. The hair removal system utilizing a reusable non-strip based member for applying, spreading and/or removing a hair removal compound.

According to one aspect, the hair removal system for use in removing unwanted hair by mechanical removal of the hair from the hair follicle, utilizing a sugar based natural wax which can be applied to a target hair removal area such that the sugar based natural wax can be warmed on contact with the users skin to bond with the hair to be removed and then quickly removed from the skin to remove not only the hair removal compound, but also hair which has bonded thereto, the hair removal system including a volume of hair removal compound configured to bond with hair upon application to the user's skin. A reusable flexible member adapted to scoop, apply and/or remove the volume of hair removal compound with respect to a target hair removal area.

A method for utilizing a hair removal system for use in removing unwanted hair by mechanical removal of the hair from the hair follicle, utilizing a sugar based natural wax which can be applied to a target hair removal area such that the sugar based natural wax can be warmed on contact with the users skin to bond with the hair to be removed and then quickly removed from the skin to remove not only the hair removal compound, but also hair which has bonded thereto, the hair removal system comprising. Obtaining a volume of hair removal compound configured to bond with hair upon application to the user's skin. Obtaining a reusable flexible member adapted to scoop, apply and/or remove the volume of hair removal compound with respect to a target hair removal area. Scooping a bolus of hair removal compound from the volume of hair removal compound. Applying the bolus of hair removal compound to the target hair removal area utilizing the reusable flexible member. Spreading the bolus of hair removal compound to form a layer substantially covering the target hair removal area. Removing the layer of hair removal compound utilizing the reusable flexible member. As necessary reapplying the same bolus of hair removal compound to a subsequent target hair removal area.

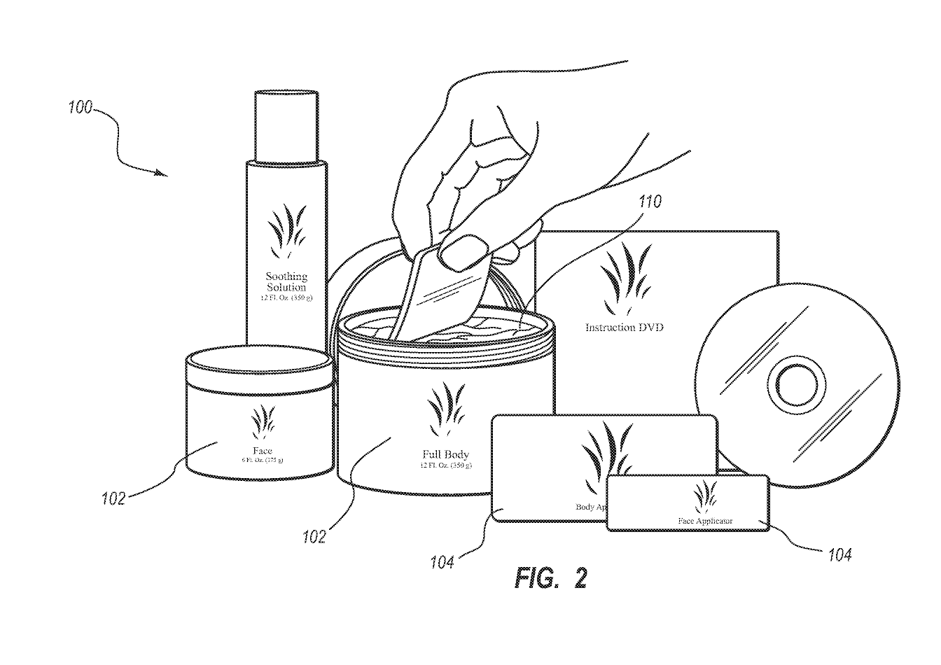

FIG. 2 illustrates components of the hair removal system of the present invention including reusable removal apparatus. In the illustrated embodiment, an improved system is depicted including a container having hair removal compound 102, a reusable apparatus 104 including a bulk removal apparatus 106 and/or a targeted reusable apparatus 108, optionally a post removal lotion or crème 109 can also be provided.

According to one illustrative embodiment of the present invention, hair removal compound 102 comprises a sugar based hair removal compound. Sugar based hair removal compounds can be desirable due to their ease of application, material and performance related properties and the fact sugar based hair removal compounds can be reusable when utilized with a reusable removal apparatus 104. For example, sugar based hair removal compounds 102 tend to be slightly more solid at room temperature. According to illustrative embodiments of the present invention, sugar based hair removal compounds can be similar to caramel or a butterscotch ice cream topping at room temperature.

The reusable removal apparatus 104 can be utilized to obtain hair removal compound 102 from the container and apply the hair removal compound to the patients skin. Once the hair removal compound 102 has been applied, the reusable removal apparatus 104 are then utilized to remove the compound and simultaneously the hair from the target location 14 of the user. The sugar based hair removal compound 102 can then be reapplied, discarded, or returned to container for future use. The reusable removal apparatus 108 can be reused immediately, cleaned for future use and stored. The benefits of a reusable removal apparatus 108 are many. Ease of use, multiples uses including scooping compound, application to user, and removal of material, and simple and easy storage of a small yet attractive component.

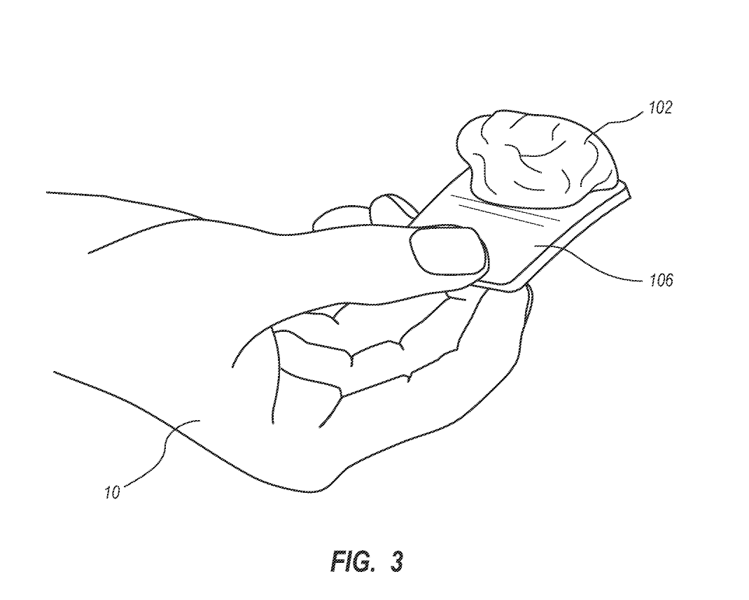

FIG. 3 illustrates a user obtaining a bolus of sugar based hair removal compound in preparation for employing hair removal methodology. As will be appreciated by those skilled in the art, the material properties and room temperature can be selected and slightly modified to provide desired consistency. Typically, the material properties will involve the ability to remove a bolus 110 of sugar as depicted in FIG. 3. This sticky, yet malleable material can be removed in a bolus that is sized for a particular treatment area. For example, a bolus that is roughly the size of a golf ball or ping-pong ball may be desired to apply to the user's leg. A slightly larger sized bolus may be desired for a particularly large treatment area. A smaller sized bolus would be desired for a smaller treatment area. For example a marble sized bolus may be desired to treat a user's upper lip.

Bulk removal apparatus 104 is depicted. Bulk removal apparatus 104 in the illustrated embodiment is roughly the size of a credit card. Bulk removal apparatus 104 will typically be formed from a resilient but smooth material to facilitate contact with hair removal compound 102. In the illustrated embodiment, bulk removal apparatus includes a first end, a second end, a first lateral extremity and a second lateral extremity. A first side and a second side are also depicted. A user will typically grasp bulk removal apparatus 104 on one of first end and second end by grasping the bulk removal apparatus on first and second lateral sides. The other of the first and second end is then dipped into the container such that a leading edge can be utilized to scoop a volume of material. The smooth first and second sides are smooth to form adhesive like contact with the material, which then can be applied to the user's skin.

A targeted reusable removal apparatus 108 is also applied in a manner facilitating use of a smaller reusable removal apparatus 104 when desired.

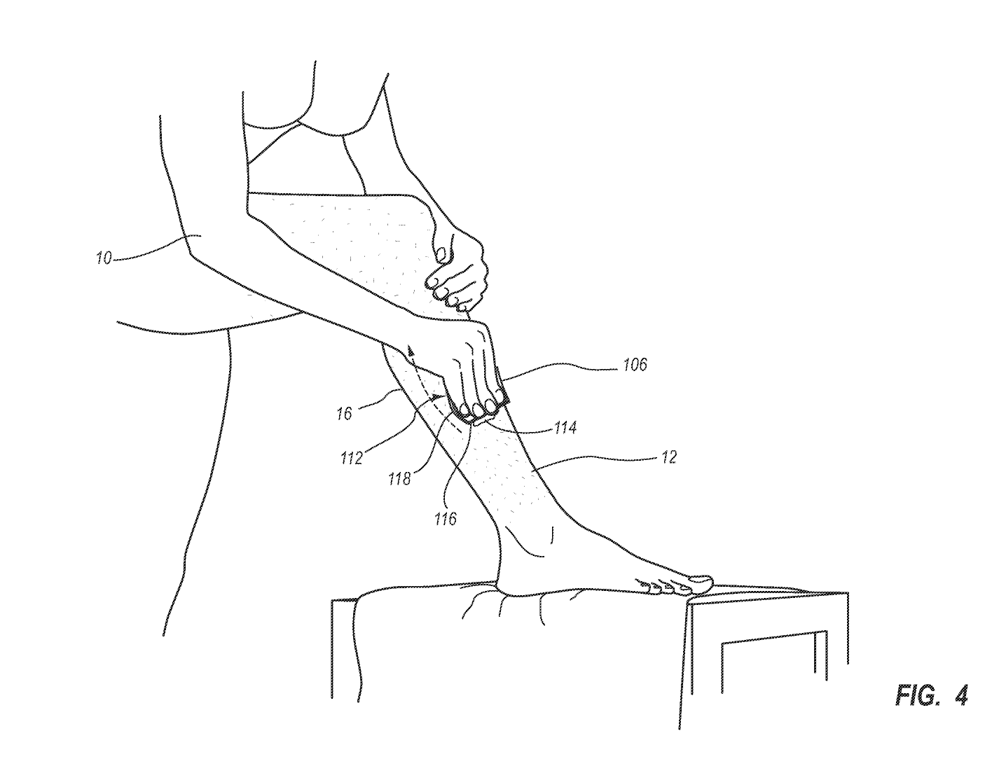

FIG. 4 is a perspective view illustrating a user applying hair removal compound to a target location for hair removal. In the illustrated embodiment, hair removal compound 102 is positioned largely on one of the lateral sides of reusable removal apparatus 104. The user simply spreads the collected volume of compound to the target location 112. Using one or several swiping motions, the material can be applied to target location 112. In this manner a consistent and uniform layer of material are applied to the user's target location 112.

A volume of hair 113 associated with target location is depicted. Hair can be regrowing stubble, or unshaved longer hair. Additionally finer hair or more coarse hair may be treated. As the hair removal compound 102 is applied to target area, the hair removal compound will begin to warm due to contact with the user's skin. The warming of the compound is desired. As a result, users are typically encouraged to apply sugar in a cool place without first warming of compound. Though for sensitive areas, the removal compound 200 may be warmed slightly to facilitate spreading.

Subsequent to contact with the patient's skin, the compound will warm and become slightly more fluid. As a result, the removal compound 200 will bind more readily with hair 113 at the target location.

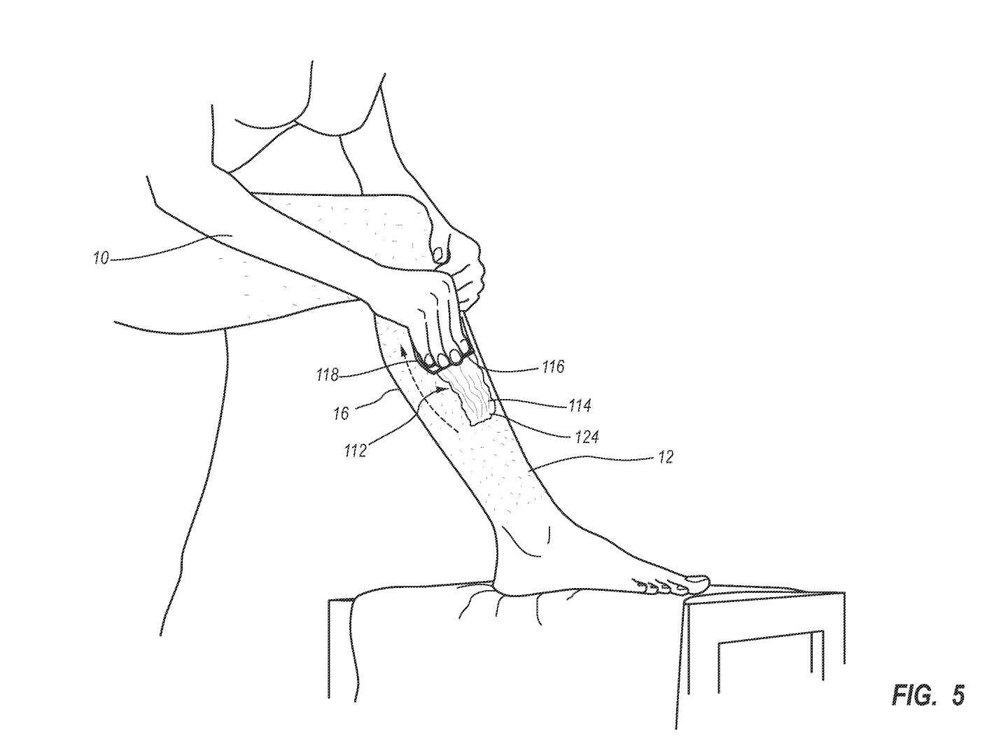

FIG. 5 illustrates a layer of removal compound that has been spread across the target location. In the illustrated embodiment, the hair removal compound 102 has been applied in a desired manner forming a spread layer 114 of removal compound 102. All or a portion of the target location 14 has been covered with removal compound 102. According to one illustrative embodiment, a somewhat small amount of time has elapsed since the original application of removal compound 102. In one illustrative embodiment, the step of applying the bolus 110 of removal compound 102 such that it forms a spread layer 114 has already been performed. As a result, the time, which elapses between forming, spread layer 114 and removal of spread layer 114 can be minimal to almost immediate.

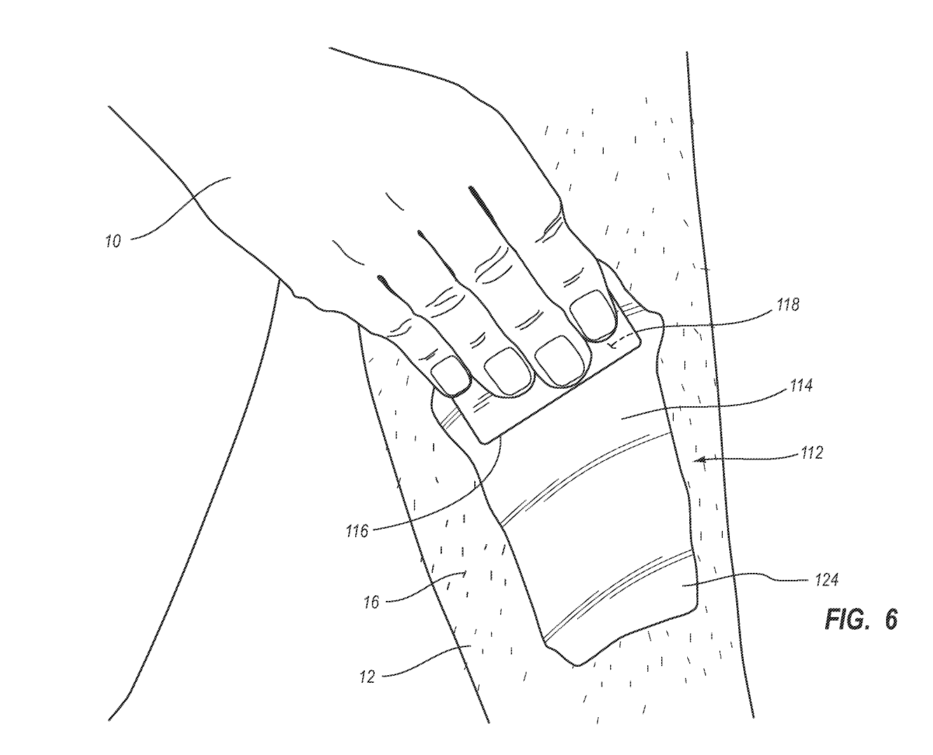

FIG. 6 depicts positioning of the reusable removal apparatus in preparation for removal of hair from the target area. In the illustrated embodiment, the leading edge of the second end of bulk removal apparatus 106 is positioned over a first portion of spread layer 114. A portion of one lateral side of bulk removal apparatus 106 is positioned in contact with spread layer 114. This positions the bulk removal apparatus 106 in a desired position to begin removal of spread layer 114 of removal compound 102.

As previously discussed, a portion of spread layer 114 is positioned in contact with the patient's skin. The warming of spread layer 114 facilitates bonding or other adhesive or mechanical interaction with hairs in a target removal location 14. As a result, removal of spread layer 114 can occur quickly and without requisite cooling periods of other systems. Similarly, application of a disposable strip is unnecessary. Instead, subsequent to application of removal compound 102 utilizing removal apparatus 106 in a spread layer 114, removal apparatus 106 can be positioned to a the position depicted in FIG. 6 with little or no time or effort. As a result, the combination of removal compound and removal apparatus 104 provides an intuitive and quick and simple system for application and removal of hair from the user 10.

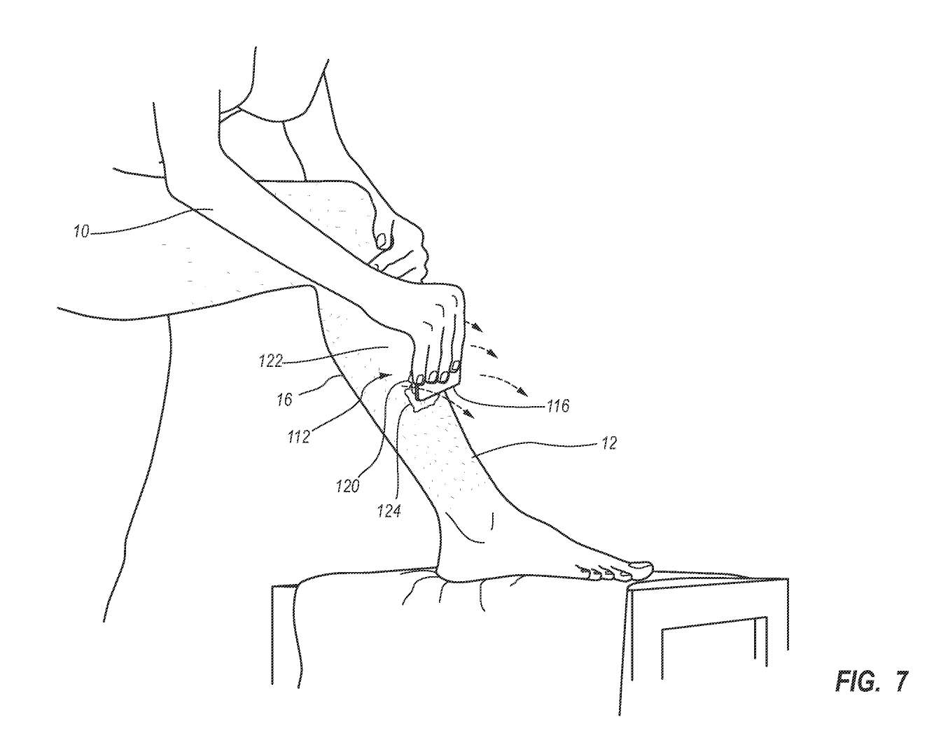

FIG. 7 illustrates removal of all or a portion of hair removal compound utilizing the reusable removal compound. In the illustrated embodiment, the user begins the process of removal of spread layer 114. In a quick motion, the user simply moves the removal apparatus laterally in a direction along the length of the spread layer 114. This removes the spread layer 114 and hair that has bonded with spread layer 114. In one illustrative embodiment, a series of quick flicking motions are utilized. Each “flick” involves slightly moving laterally along the spread layer 114 while lifting slightly to provide a desired amount of clearance over the spread layer. Subsequent to a flick, the user simply pressed the removal apparatus 108 and any removal compound 102 attached thereto against the spread layer 114. A subsequent one or more “flicks” results in removal of the entire spread layer.

This entire sequence of flicks can occur in a very short amount of time, even less than one or two seconds. As a result, not only is the system intuitive, simple and straightforward to utilize, but the removal compound 102 which remains can then be immediately reapplied to a subsequent target area and reutilized. According to one embodiment of the present invention, the removal compound is applied against the direction of hair growth and removed in the direction of the hair growth. This provides a more comfortable and less painful removal process. As a result, less irritability may be experienced by the patient leading to an improved hair removal experience.

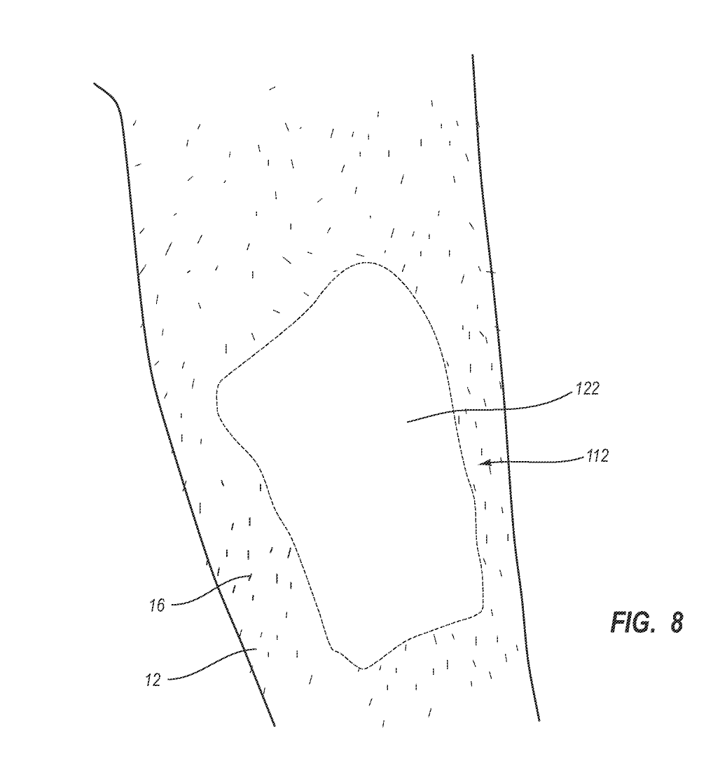

FIG. 8 illustrates a section of the user's body where hair has been removed utilizing the hair removal compound and the reusable removal apparatus. In the illustrated embodiment, the effects of the quick removal of spread layer 114 of removal compound 102 utilizing reusable removal apparatus 104 is depicted. In other words, hair 113 originally visible on target location 112 has been removed leaving a hair free and smooth skin. Additionally, by removing the hair along the grain of the hair, unnecessary discomfort can largely be avoided. This minimizes redness, bumps and other irritation over existing systems. Any artifact removal compound 102 can simply be swabbed up utilizing bolus 102 and/or by washing with warm soap and water. The user can then apply a skin moisturizer or other lotion to leave the legs smooth and soft.

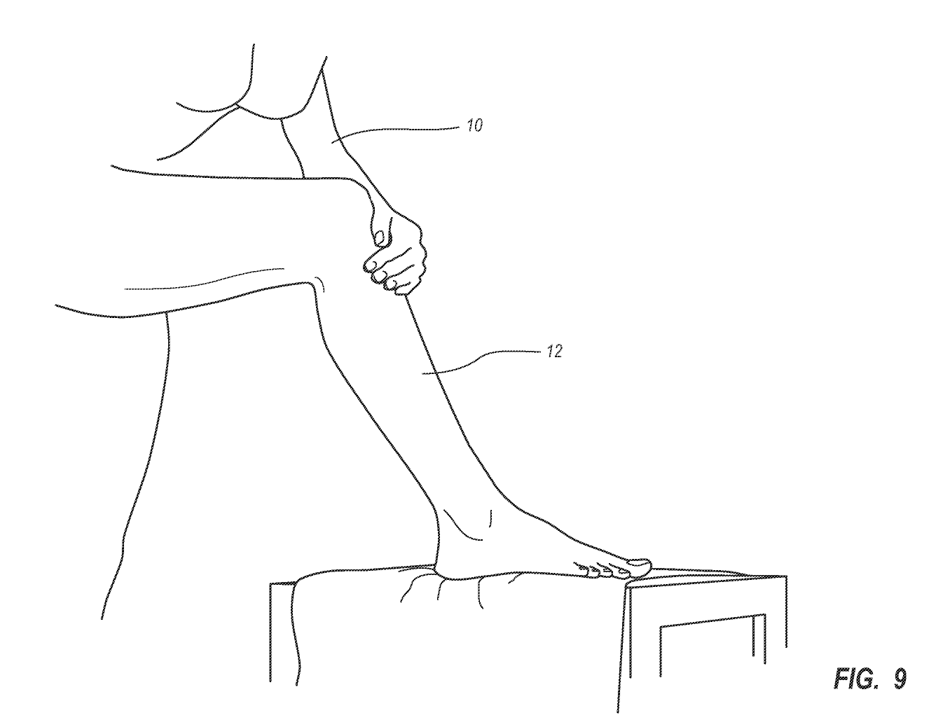

FIG. 9 depicts a user's leg that has been treated resulting in complete hair removal. In the illustrated embodiment, one or a plurality of target locations on the user's legs have been treated leading to hair free legs. In one illustrative embodiment, the same bolus 110 of removal compound 102 is utilized. As a result, not only are disposable strips of other systems not needed, but even greater efficiencies can be realize by not having to dispose of removal compound stuck to disposable strips. Instead, removal compound can quickly and simply be reapplied utilizing removal apparatus 104. In this manner, a number of target locations can be quickly treated in successive and repetitive steps utilizing a single reusable removal apparatus 106. Not only does this save time and money, the user will be more likely to be able to find the neatly organized components of the hair removal system and be willing to conduct subsequent removal steps in the future.

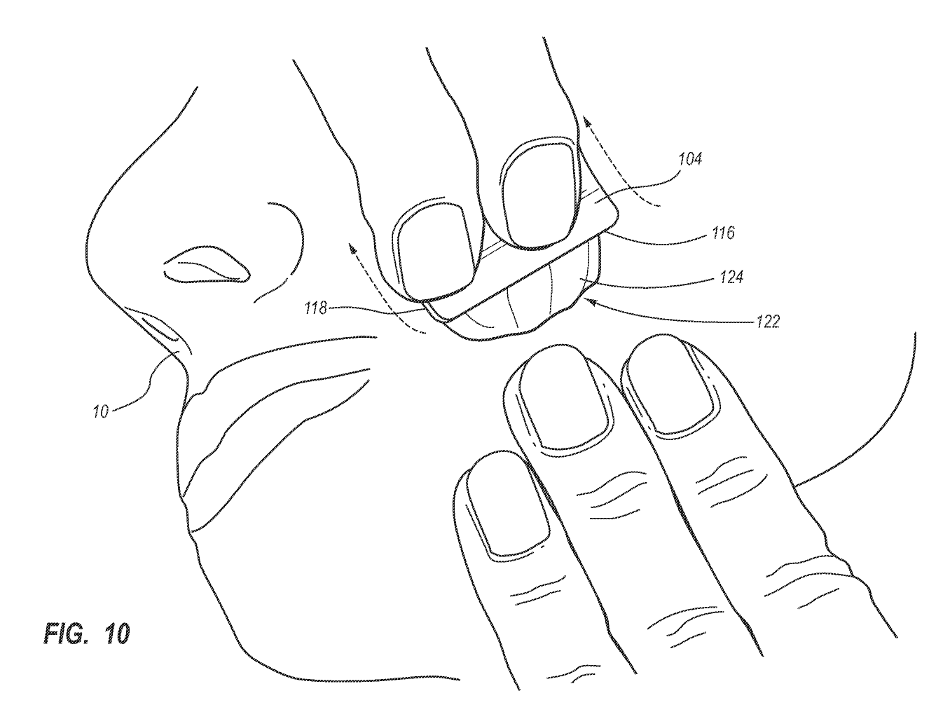

FIG. 10 depicts a targeted reusable hair removal apparatus being utilized on a user's face. In the illustrated embodiment, use of targeted reusable removal apparatus 108 is depicted. The size and shape of targeted removal apparatus 108 allows a user to reach areas that would not be convenient with bulk removal apparatus 106. However, many of the steps and principles involved with removal depicted in FIGS. 2-8 also will apply to targeted reusable removal apparatus 108 as will be appreciated by those skilled in the art.

A variety of types and configurations of hair removal based systems can be utilized without departing from the scope and spirit of the present invention. For example, in one embodiment, a metal or non-plastic reusable removal apparatus is utilized. In another embodiment, the shape, length or size of the removal apparatus can be varied. In another embodiment, a separate applicator is utilized separate from the removal apparatus. In yet another embodiment, a hair removal compound other than a sugar based hair removal compound is utilized. In another embodiment, disposable applicators and/or non-strip removal apparatus are utilized.

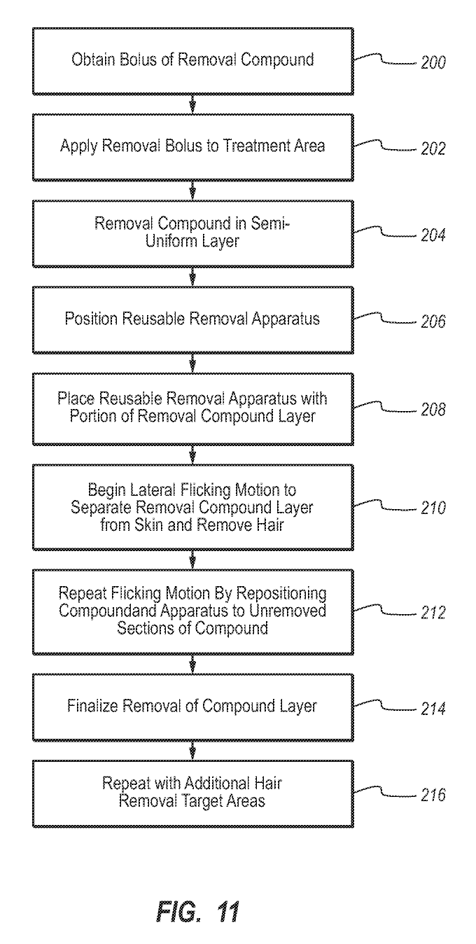

FIG. 11 illustrates a method for utilizing a hair removal compound with a reusable removal apparatus. In the illustrated embodiment, a practitioner obtains bolus of removal compound in a step 200. The practitioner applies the bolus of removal compound to the treatment area in a step 202. The practitioner the spreads the removal or otherwise applies the removal compound in a semi-uniform layer in a step 204. The practitioner then positions the reusable removal apparatus in a step 206. The practitioner then places the reusable removal apparatus with a portion of the removal compound layer in contact with a portion of the reusable removal apparatus in a step 208.

Once the compound is in contact with the reusable removal apparatus, the user can begin removing the removal compound layer and the hair, utilizing lateral, downward or other removal motions in a step 210. The user then can repeat the removal motions as needed to remove additional sections of compound in a step 212. The user can then finalize removal of the entire compound layer to remove all, or substantially all of the hair from the target hair removal area in a step 214. The user can the repeat the steps of applying and removing in additional hair removal target areas in an optional step 216.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

### NUMBERING

User 10
Leg 12
Target Location 14
Hair 16
Wax 18
Disposable Strips 20
Hair Removal System 100
Sugar Based Hair Removal Compound 102
Reusable Removal Apparatus 104
Bulk Removal Apparatus 106
Targeted reusable Removal Apparatus 108
Bolus 110
Target Location 112
Hair 113
Spread Layer 114
Leading edge 116
Contact surface 118
Removed portion 120
Treated area 122
Reusable bolus 124
Obtain bolus of removal compound 200
apply removal bolus to treatment area 202
spread removal compound in semi-uniform layer 20
position reusable removal apparatus 206
place reusable removal apparatus with portion of removal compound layer 208
Begin lateral flicking motion to separate removal compound layer from skin and remove hair 210
repeat flicking motion by repositioning compound and apparatus to unremoved sections of compound 212
finalize removal of compound layer 214
repeat with additional hair removal target areas 216.

1. A hair removal system for use in removing unwanted hair by mechanical removal of the hair from the hair follicle, utilizing a sugar based natural wax which can be applied to a target hair removal area such that the sugar based natural wax can be warmed on contact with the users skin to bond with the hair to be removed and then quickly removed from the skin to remove not only the hair removal compound, but also hair which has bonded thereto, the hair removal system comprising;
a volume of hair removal compound configured to bond with hair upon application to the user's skin;
a reusable flexible member adapted to scoop, apply and/or remove the volume of hair removal compound with respect to a target hair removal area.

2. The hair removal system of claim 1, wherein the reusable flexible member comprises a flat member.

3. The hair removal system of claim 1, wherein the reusable flexible member comprises a plastic card shaped member.

4. The hair removal system of claim 1, wherein the reusable flexible member comprises a bulk removal apparatus.

5. The hair removal system of claim 1, wherein the reusable flexible member comprises a targeted reusable removal apparatus.

6. The hair removal system of claim 1, wherein the hair removal compound comprises a sugar based removal composition.

7. The hair removal system of claim 1, wherein the hair removal compound comprises a non-sugar based removal composition.

8. The hair removal system of claim 1, wherein the hair removal compound comprises an adhesive mixture which is adapted to form an adhesive or other mechanical connection with the hair to be removed.

9. The hair removal system of claim 1, wherein the hair removal compound is adapted to remove hair utilizing a mechanism other than mechanical bonding.

10. A method for utilizing a hair removal system for use in removing unwanted hair by mechanical removal of the hair from the hair follicle, utilizing a sugar based natural wax which can be applied to a target hair removal area such that the sugar based natural wax can be warmed on contact with the users skin to bond with the hair to be removed and then quickly removed from the skin to remove not only the hair removal compound, but also hair which has bonded thereto, the hair removal system comprising;
obtaining a volume of hair removal compound configured to bond with hair upon application to the user's skin;
obtaining a reusable flexible member adapted to scoop, apply and/or remove the volume of hair removal compound with respect to a target hair removal area;
scooping a bolus of hair removal compound from the volume of hair removal compound;
applying the bolus of hair removal compound to the target hair removal area utilizing the reusable flexible member;
spreading the bolus of hair removal compound to form a layer substantially covering the target hair removal area;
removing the layer of hair removal compound utilizing the reusable flexible member; and
as necessary reapplying the same bolus of hair removal compound to a subsequent target hair removal area.
